(19) United States
(12) Patent Application Publication
(10) Pub. No.: US 2026/0345849 A1
(43) Pub. Date: Nov. 5, 2026

(54) PROCESS FOR THE PREPARATION OF A SCENT DIFFUSER CARTRIDGE, CARTRIDGE OBTAINED THEREBY AND DIFFUSER APPARATUS USING SAID CARTRIDGE

(71) Applicant: VENTURE VENTIDUE SRLS, Sesto San Giovanni (MI) (IT)

(72) Inventor: Simone FURIATO, Sesto San Giovanni (MI) (IT)

(21) Appl. No.: 19/100,039

(22) PCT Filed: Aug. 1, 2022
(86) PCT No.: PCT/IB2022/057130

§ 371 Date: Jan. 30, 2025

(51) Int. Cl. A61L 9/04

(52) U.S. Cl. CPC A61L 9/046; A61L 9/042; A61L 2209/133

(57) ABSTRACT

A process for preparing a scent diffuser cartridge includes providing and sieving granules of a material of natural mineral rock origin, placing a predetermined quantity of over-sieve granules into a container, diffusing, into the container, a liquid scent substance in a quantity proportional to the quantity of granules present in the container, closing the container, letting the granules in the container stand for a predetermined time to cause the granules to absorb the scent substance diffused in the container, and checking whether any residues of liquid are present inside the container. If the absence of residues is detected, a mixture containing 60% to 90% granules and 10% to 40% over-sieve granules are combined in a vessel and the mixture is permitted to stand. A casing is provided with an air-permeable porous wall, a quantity of the mixture is placed into the casing, and the casing is closed and packaged.

### TECHNICAL FIELD

The invention relates to a process for the preparation of a scent diffuser cartridge, the cartridge obtained thereby and a diffuser apparatus using said cartridge.

BACKGROUND ART Methods currently available on the market for diffusing scents and fragrances can be divided into some macro-categories:
methods based on combustion (candles, incenses, catalytic lamps);
methods based on aerosol technology, which exploits the Bernoulli principle or ultrasounds;
methods based on spraying and/or nebulization;
methods using synthetic solid capsules or gels (for example, EVA, PET, silicones);
methods using sticks immersed in a liquid.

Each of these methods provides for using a diffuser device, specifically designed for being used with the corresponding method.

According to prior art, all the aforesaid methods require a mixture comprising a scent substance, which is usually produced in the laboratories of essence companies, typically an essential oil, or a fragrance, and a substance suitable to promote the operation of the method and corresponding device. In other words, the scent substance is not used as it is, but it is mixed with a carrier suitable for the method and the device for which the mixture is intended. Said carrier must be capable of promoting the diffusion of the scented fragrance in the air, but, on the other hand, it has a content that sometimes can be harmful to humans if inhaled for a long time and alters the scent characteristics of the substance with which it is mixed.

In prior art methods and devices, the quality of what is perceived by smell is therefore always less than 100% of the quality associated with the fragrance that was initially created by the laboratories of the essence companies. In fact, the maximum concentration which these aforementioned methods can reach is always less than 25% essential oil/fragrance within the mixture used in the device, and the rest is a mixture necessary for the operation of the chosen device.

The most widely marketed diffuser apparatuses for diffusing fragrances use the technology of nebulization, which occurs as a result of the Venturi effect. In order to be able to nebulize an essence, these systems can receive mixtures that contain DPG (dipropylene glycol)/water/alcohol and fragrance oil. In most cases, these mixtures never exceed 25% fragrance oil concentrate because the nozzles that are to nebulize by means of the Venturi effect would not be able to nebulize the liquid droplets and create the mist effect necessary to diffuse the fragrance. Some devices also use the ultrasonic system (aerosol type), and even in this case the mixture should not be too concentrated in essential oil, and there is currently a maximum limit of 25% concentration of fragrance oil.

Recently, there has been a mass spreading of systems nebulizing scents into the air. Nebulization has become very fashionable as it allows a large amount of “scented” substance to be spread in the air and is capable of reaching very large spaces in a short time, even outdoors.

This effect is an advantage from a technical and commercial point of view, but it is contrary to compliance with the criterion of “awareness” for consumers who, when they are in the environment where diffusion takes place, have no choice and are forced to consume a product simply by breathing.

It is precisely breathing that unintentionally determines the intake of these aromas that reach parts of our bodies, without the end consumer being aware of it.

Through combustion, nebulization or spraying, tiny particles of substances are created that humans are able to ingest and inhale, carrying them to the lung alveoli.

FIG. 1 is a diagram devised by ISPRA (Istituto Superiore per la Protezione e la Ricerca Ambientale italiano=Italian Institute for Environmental Protection and Research), which graphically shows the size of the dusts diffusing into the human respiratory system.

The current practice of diffusing, into the environment occupied by living beings, mixtures of scent substances, which contain a small percentage of essential oil/fragrance carried by compounds necessary for diffusion and which can penetrate to the alveoli of human beings, also arouses interest in the medical world, which is increasingly studying the effects of particulate matters (PM10/PM2.5).

One of the main objects of the invention is therefore to overcome the drawbacks of prior art, and further provide a solution to the problem of how to reduce the risks for the health of living beings deriving from the diffusion of scent substances in environments.

A further object of the invention is to provide a solution to the aforementioned problem that is also effective in retaining the olfactory properties of scent substances created by the laboratories of essence companies.

Another object of the invention is to provide a cartridge for olfactory stimulation by means of the diffusion of scent substances that is harmless to human health and therefore does not release harmful substances into the environment.

Not least object of the invention is to provide a process for preparing a scent diffuser cartridge, a cartridge obtained by said method, and a diffuser apparatus using said cartridge, which can be used industrially and manufactured in a cost-effective way and thus lend themselves to large-scale production.

### SUMMARY OF INVENTION

The above-mentioned and other objects are achieved with the process for preparing a scent diffuser cartridge, said process essentially comprising the steps of:
providing a quantity of granules of a material of natural mineral rock origin, capable of retaining a scent substance;
subjecting the material in the form of granules to sieving;
placing a predetermined quantity of over-sieve granules into a container;
diffusing, into the container, a liquid scent substance in a quantity proportional to the quantity of granules present in the container;
closing the container;
letting the granules in the container stand for a predetermined time in order to cause the granules to absorb the scent substance diffused in the container;
checking whether any residues of liquid are present inside the container;
if, at the previous step, the presence of residues of liquid has been detected, letting again the granules in the container stand for a predetermined time and possibly repeating the previous step;
if, at the previous step, the absence of residues of liquid has been detected, providing, in a vessel, a mixture containing 60% to 90% granules having absorbed the scent substance and obtained with the previous steps, and 10% to 40% over-sieve granules;
letting the thus-obtained mixture stand;
providing a casing provided with an air-permeable porous wall;
placing a quantity of said mixture of granules into the casing;
closing the casing, thereby obtaining a scent diffuser cartridge;
packaging the cartridge in order to put it on the market.

Advantageously, according to the invention, the cartridge does not contain any liquids, is based on a material of natural origin rather than of synthetic origin, and the material inside the cartridge cannot come into contact with the user's hands because it is protected by the porous casting.

Advantageously, the process according to the invention allows making a diffuser cartridge based on a solid substance of natural origin, which is capable of absorbing a scent substance as produced by essence companies, in a quantity higher than that of synthetic mixtures of plastic origin known to date (for example, EVA, PET, silicones, etc.), and which, once it has exhausted its capacity of “retaining” the fragrance, allows easy discarding thereof.

According to the invention, the material of natural mineral rock origin, capable of retaining a scent substance, is selected from vermiculite, expanded vermiculite, perlite, hydroponic perlite, expanded perlite, agri-perlite, or a mixture thereof.

According to the invention, said material is preferably hydroponic perlite or expanded perlite, also known commercially under the name of agri-perlite, obtained from siliceous volcanic rock by means of a thermal process.

These materials in the form of inert natural granules have an extremely high capability of absorbing and retaining liquids and can be used together and/or separately depending on the specific weight of the liquid, for example, essential oil, and the end-use thereof.

In a preferred embodiment of the invention, the material in the form of granules has a low content of silicon, preferably comprised between 40 and 60%, and more preferably between 20 and 40%.

According to a particular embodiment of the invention, the scent substance, for example an essential oil produced by an essence company, is subjected to a suitability tests in order to identify the most suitable type of material of natural mineral rock origin, or a mixture of such materials.

According to this particular embodiment of the invention, the absorption capability of the mineral rock is tested in the following way: one part of scent substance, for example fragrance oil, and one part of granules of mineral rock (1.0 g oil/1.0 g rock). This preliminary testing step is performed on various types of material of natural mineral rock origin. For example, the test is carried out with vermiculite and scent substance (for example fragrance oil), with expanded vermiculite and scent substance, with perlite and scent substance, with agri-perlite or expanded perlite, or with a mixture of these materials with the scent substance. After assessing the absence of oil leakage in at least one of these combinations, the most suitable mineral is selected from vermiculite, perlite, agri-perlite, etc . . . or a mixture thereof.

According to the invention, the choice of the most suitable mineral is further determined by the final volume to be occupied by the granules of mineral material inside the cartridge, which volume might vary according to the applications and might be subjected to forced air or natural ventilation.

In the case of essential oils, the absorption capability of expanded vermiculite and agri-perlite is essentially 1:1, i.e. they can absorb as much as their weight.

Some advantages identified in connection with these two natural materials are listed below: Lightweight (volumetric mass 35/150kg/m³);
Fire reaction Class A1 (non-combustible according to Italian Ministerial Decree 10.3.2005);
High melting point: 1260/1343 °C;
Essentially neutral PH 6.5/7.5;
Insoluble in water, organic acids and mineral acids;
Nontoxic;
Inert.

Advantageously, the absence of dispersed liquid inside the scented cartridge increases the possibility of transport, storage and marketing worldwide. In addition, for the same reason, the safety level in case of “breakage” of the cartridge is very high. The use of a liquid-free cartridge, indeed, increases the safety level in terms of flammability and fire induction.

Advantageously, in addition, due to the fact that the scent substance within the cartridge is entirely retained by the inert material of natural origin and is released only when the cartridge is subjected to a flow of forced and/or natural air, optimal energy efficiency is ensured.

Advantageously, the use of a material in the form of granules for the cartridge makes it possible to manufacture cartridges with different fragrances inside them, without the various scents becoming contaminated and without the various essential oils and/or fragrances becoming mixed with one another, and thus prevents non-compliance with the regulations of IFRA (International Fragrance Association) and REACH (Registration, Evaluation, Authorisation and Restrictions of Chemicals) due to non-controllable chemical reactions. According to the invention, the over-sieve material preferably comprises granules having a diameter size between about 4 and 10 mm, and even more preferably a diameter size between about 4 and 5 mm.

Advantageously, the sieving step provided in the process according to the invention allows obtaining an over-sieve of granules having a size larger than that of the material usually available naturally and commercially. The larger size of the granules allows obtaining a base material for the making of the diffuser cartridge that is substantially free from dusts with a size such that they can penetrate deeply into the human respiratory system, and particularly to the level of the alveoli.

Advantageously, according to the invention, sieving in order to obtain larger granules does not derive only from the need to avoid the presence of dusts in the material for the making of the diffuser cartridge, but it also associated with the “finding” that the behavior of the base material, having a natural mineral rock origin and capable of retaining a scented substance and, in particular, the behavior of perlite and perlite for agricultural use, also known as agri-perlite, exhibits higher performance in terms of capability of absorbing the scent substance and thus diffusing it during use, when the granules are larger.

According to the invention, the scent substance is capable of releasing a scented fragrance in the air and is preferably alcohol-free and may optionally contain a carrier, such as dipropylene glycol (DPG), while preferably having no additional solvents or water.

According to the invention, the scent substance is preferably an essential oil or a mixture of essential oils.

Even more preferably, according to the invention, the scent substance is a mixture created by an essence company, or fragrance laboratory, and contains therein the extract of essential oil with the required olfactory notes (for example, in the case of lavender, an extract of lavender essential oil, and the same applies also to a root such as cedar wood or to vanilla). This mixture is an oil solution complying with all the regulations of IFRA and REACH, i.e. it does not contain, in absolute values, the quantities prohibited by law of some substances indicated as dangerous substances by ECHA (European Chemical Agency) in its SVHC (Substances of Very High Concern) list.

According to the invention, the quantity in weight of scent substance diffused in the container is preferably about 85-95% of the weight of the material present in the container, and even more preferably about 90% of the weight of the material present in the container. According to the invention, the container is preferably at least transparent, and even more preferably it is made of glass and/or steel.

According to the invention, the container is preferably closed by means of a closure made of a non-absorbent material, preferably glass, or an impermeable material, and is preferably kept out of direct light.

According to the invention, preferably, the step of diffusing the scent substance in the container and the step of closing the container are carried out without moving the container, and accordingly without moving the content of the container.

According to the invention, the scent substance is diffused in the container preferably by gravity in the form of rain or by nebulization.

According to the invention, the container preferably consists of a container having a vertically elongated cylindrical shape and the step of diffusion by gravity in the form of rain preferably takes place from above by means of a strainer with holes having a diameter of about 1.0 mm, or through a nebulizing nozzle.

According to the invention, preferably, the granules of material in the container are let stand, at the end of the step of diffusion of the scent substance, for a time proportional to the quantity of diffused substance, preferably at a temperature comprised in a predetermined range and preferably without direct exposure to light or darkness.

According to the invention, preferably, the granules of material in the container are let stand, at the end of the step of diffusion of the scent substance, for a time of about 1 hour per each mL of liquid scent substance diffused in the container.

According to the invention, preferably, the container containing the granules of material in which the scent substance was diffused is turned upside down at the end of the standing step.

According to the invention, preferably, the step in which the granules of material inside the container are let stand after the presence of liquid inside the container has been detected after the first standing step, is carried out for a time of 30 minutes per each mL of essential oil/fragrance poured into the vessel.

According to the invention, preferably, the step in which the granules of material inside the container are let stand after the container has been closed, is carried out at a temperature between 15 and 20 °C. Lower or higher temperatures could alter the absorption times of the granules and would involve the risk of creating condensates. More precisely, below 15 °C, sediments form on some fragrances, for examples containing vanilla, woody notes and/or aromatic notes, which sediments cannot be absorbed by the granules, whereas above 20°, with fragrances for example based on flowers, citruses and in some cases with presence of vanilla and/or aromatic notes, there is an onset of loss from the olfactory point of view and an undesired evaporation of the so-called top notes is triggered. By “top notes” it is meant those notes formed by oils which evaporate quickly and correspondingly quickly reach the nose. They are the smell that is perceived first when smelling an essential oil and they are often also the strongest notes, which, however, vanish very quickly.

According to the invention, preferably, the casing may be rigid or flexible and is provided with an air-permeable porous wall having a filtering capacity between 40 and 70 microns, i.e. the wall can retain, inside the casing, particles with a diameter smaller than the selected range.

According to the invention, preferably, the casing provided with an air-permeable porous wall comprises a porous wall with a filtering mesh having a weight of over 60 gr/m².

According to the invention, preferably, the casing provided with an air-permeable porous wall contains a weight quantity of granules smaller than 1,0 gr.

According to the invention, the casing may have any shape and also have the shape of a flat sheet, a cylindrical, cubic, parallelepiped, prism-like shape and have a minimum development in height in the order of 1 mm.

According to the invention, optionally, the cartridge is provided with an identification label, for example bearing a bar code, a QR code, or comprising a RFID (Radio Frequency Identification) TAG containing digital information associated, for example, with the content of the cartridge, the intended use, the recipient customer, the duration, etc.

The cartridge may be made in various forms and bear the TAG on its filtering surface. In a particular embodiment, the TAG is applied at the cap or closure of the cartridge.

According to the invention, the information contained in the identification label further comprises, preferably, an unambiguous identification string allowing unambiguous identification of the cartridge.

Advantageously, according to this particular aspect of the invention, the TAG associated with the cartridge also allows re-using the cartridge with a compound identical to the one of its first use, so as to obtain actual recycling of the material.

According to the invention, preferably, the label can be read only by means of a proprietary reader or software, developed ad hoc and made non-compatible with other systems available on the market. The use of a TAG or other digital technology on the cartridge further allows managing, by means of a software, the energy efficiency and proper use of the cartridge at the desired place, thus avoiding wrong uses of improper scents at places other than the intended ones (for example, smell of a food/beverage note in an area where an eau de toilette is to be diffused).

The cartridge may be made in various forms and bear the TAG on its filtering surface. In a particular embodiment, the TAG is applied at the cap or closure of the cartridge.

Furthermore, the casing of the cartridge may be rigid or flexible.

The cartridge may have any shape, related to the mode of operation of the same in order to diffuse a scent, the place and the type of use/installation, as well as to its mode of diffusion in the environment (forced air/natural air).

The cartridges can be used either in diffuser devices with natural circulation of air, or in devices in which air circulation takes place in a forced manner, for example by means of a fan.

With these two modes, it is possible to manufacture diffusers ad hoc, capable of ensuring entrance of air and exit thereof after passing through the compound containing the scented granule.

According to the invention, preferably, the mixture inside the vessel comprises 90% granules in which the scent substance has been diffused in the preceding steps of the process according to the invention and 10% granules coming from the over-sieve and not subjected to the steps of diffusion of the scent substance.

According to the invention, in an embodiment, the vessel comprises per each 10 g of mixture: 8 g of a compound processed by absorbing the essential oil/fragrance and 2 g of inert granule (selected from perlite and/or vermiculite) sieved between 5 and 8 mm that has not been subjected to absorption.

According to the invention, preferably, the step of letting the mixture stand in the vessel is carried out for a time of four hours per each ten grams of compound obtained.

According to the invention, preferably, the casing of the cartridge is made of a filtering synthetic material or of filtering natural fibers.

Advantageously, according to the invention, the cartridge obtained is capable of diffusing a scent substance in the environment, without having to use further compounds with properties different from those mentioned in the safety data sheets of the fragrances developed by the chemical laboratories of essence companies, which, as is known, comply with the regulations of IFRA and REACH as well as with all the indications about allergens for those fragrances intended for air-fresheners.

According to a particular aspect of the invention, the step of diffusing the scent substance in the container in which the material is received can take place at a pressure lower than the atmospheric pressure, under vacuum, in order to promote and improve the absorption of the scent substance into the granule of material. The application of vacuum, when provided for, can be followed by a step in which the pressure inside the container is increased above the atmospheric pressure in order to further increase the absorption of the oil into the granule. According to a further particular aspect of the invention, the material can be subjected to an abatement treatment in order to remove possible bacteria/microorganisms. This abatement step, when provided for, may preferably be carried out on the material immediately before the step of diffusing the scent substance. In addition, a further abatement step can also be carried out downstream of the step of diffusing the scent substance into the material.

### BRIEF DESCRIPTION OF DRAWINGS

A preferred embodiment of the invention will be described below with reference to the annexed drawings, in which:

FIG. 1 is a diagram devised by ISPRA (Istituto Superiore per la Protezione e la Ricerca Ambientale italiano=Italian Institute for Environmental Protection and Research), which graphically shows the diffusion of dusts into the human respiratory system as a function of the size of the granules;

FIG. 2 is a schematic representation of a cartridge manufactured according to a first embodiment of the invention;

FIG. 3A is a schematic representation of a cartridge manufactured according to a second embodiment of the invention;

FIG. 3B is a schematic representation of a cartridge manufactured according to a third embodiment of the invention;

FIG. 3C is a schematic representation of a cartridge manufactured according to a fourth embodiment of the invention;

FIG. 4A is a front perspective view of the diffuser device according to the invention in a closed state;

FIG. 4B is a rear perspective view of the diffuser device of FIG. 4A;

FIG. 4C is a front perspective view of the diffuser device of FIG. 4A in an open state.

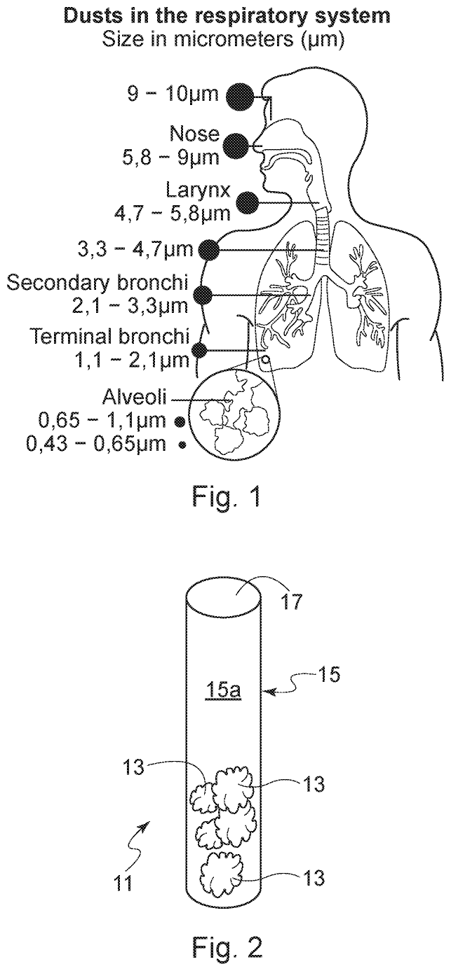

### DESCRIPTION OF EMBODIMENTS

In a preferred embodiment of the invention, the process for preparing a scent diffuser cartridge according to the invention, essentially comprises the steps of:
providing a quantity of granules of perlite for agricultural use or hydroponic perlite, ore agri-perlite having a maximum diameter size of about 4-5 mm;
subjecting the granules of perlite to sieving for separating and discarding the over-sieve part consisting of granules and dusts of small size with a diameter lower than 4-5 mm;
placing a predetermined quantity of over-sieve granules into a container;
diffusing, into the container, a liquid scent substance consisting of an essential oil, for example an extract of lavender essential oil, in a quantity proportional to the quantity of granules present in the container;
closing the container;
letting the granules in the container stand for a predetermined time in order to cause the granules to absorb the scent substance diffused;
checking whether any residues of liquid are present inside the container;
if, at the previous step, the presence of residues of liquid has been detected, letting again the granules in the container stand for a predetermined time and possibly repeating the previous step;
if, at the previous step, the absence of residues of liquid has been detected, providing, in a vessel, a mixture containing 90% granules having absorbed the scent substance and obtained with the previous steps, and 10% over-sieve granules;
letting the thus-obtained mixture stand;
providing a casing having a porous wall;
placing a quantity of said mixture into the casing;
closing the casing;
packaging the casing in order to put it on the market.

With reference to FIG. 2, this illustrates a cartridge 11 with a cylindrical shape containing granules 13 of hydroponic perlite, or agri-perlite, which have absorbed a scent substance. According to the invention, the cartridge 11 comprises a casing 15 having a porous side wall 15a.

According to the invention, the material of which the casing 15 is made may be rigid or flexible, for example a synthetic gauze with adequate porosity, preferably comprised between 40 and 70 microns.

In the shown embodiment, the cartridge 11 further comprises an identification label 17, closing one of the bases of the casing 15, the upper base in the shown embodiment. In the shown embodiment, the label 17 comprises a RFID (Radio Frequency Identification) TAG containing the digital information associated, for example, with the content of the cartridge, the intended use, the recipient customer, the duration, etc.

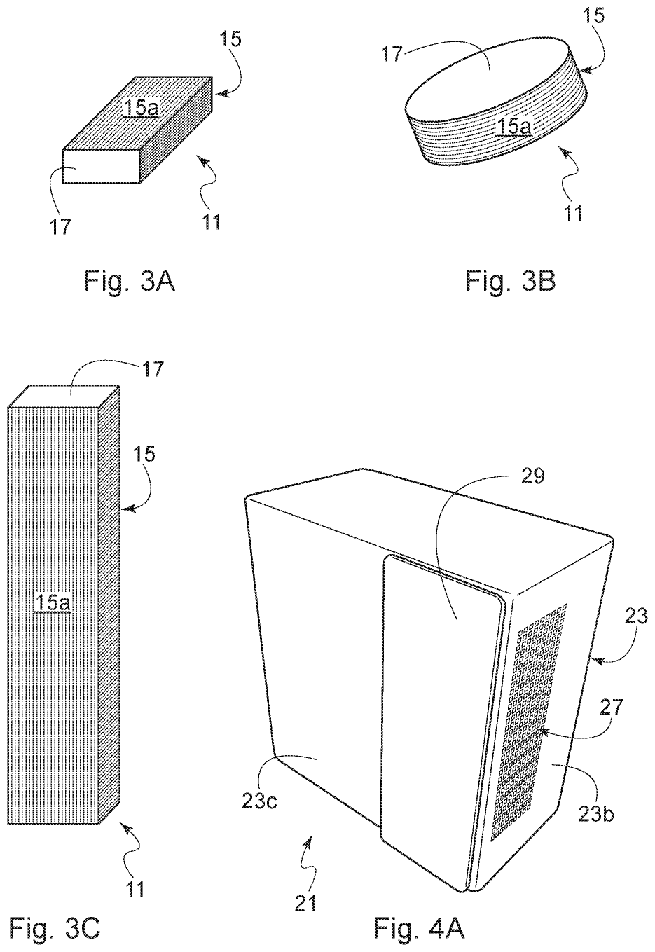

With reference to FIGS. 3A-3C, these illustrate corresponding embodiment examples of the cartridge according to the invention having the shape of a parallelepiped prism with essentially horizontal development, a cylinder with an essentially horizontal development, and a parallelepiped prism with essentially vertical development.

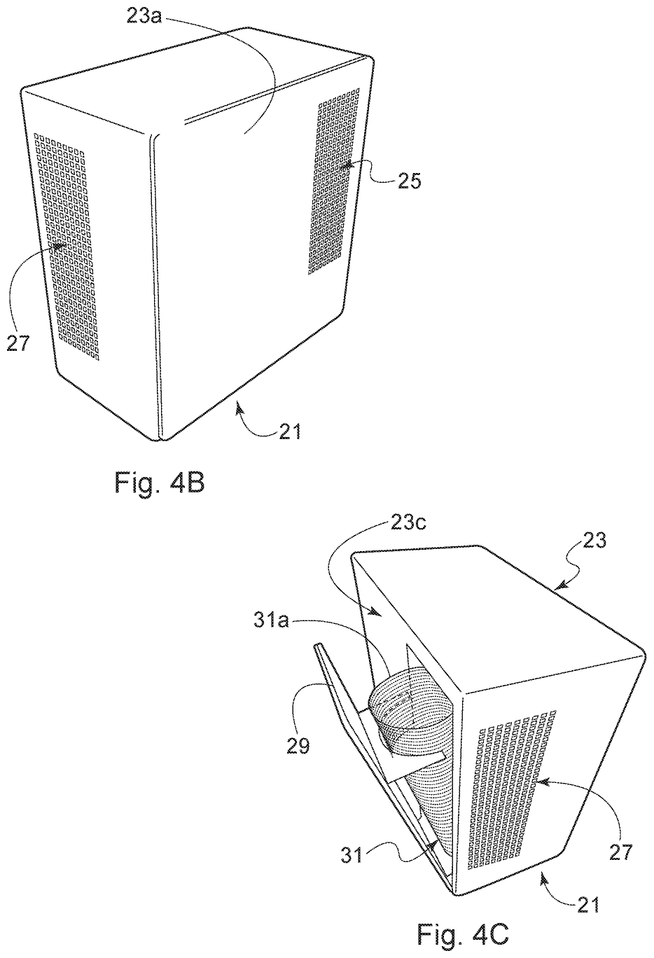

Referring to FIGS. 4A-4C, these show a diffuser apparatus 21 using a cartridge 11. The apparatus essentially comprises a casing 23, which, in the shown embodiment, has a parallelepiped shape. Inside the casing 23 there is housed a tangential-type fan taking in air from the outside through a port 25 and diffuses it into the casing 23 housing a cartridge 11 of the type obtained with the process according to the invention. The casing 23 is further provided with a port 27 for letting air exit after it flows through the inner region in which said cartridge 11 is housed. According to the invention, preferably, the port 25 and the port 27 are perpendicular to each other and the fan is thus of the tangential type, i.e. capable of taking in air in a first direction along a first axis and discharging it in a second direction along a second axis perpendicular to the first axis. In the shown embodiment, the port 25 and the port 27 are thus provided on two mutually perpendicular, adjacent sidewalls 23a, 23b of the casing 23.

Referring still to the illustrated embodiment, the front wall 23c of the casing 23 comprises an opening that can be closed by means of a corresponding cover 29. Said cover is preferably hinged at its base to the casing 23 and has, on its inner surface, a porous cylindrical receptacle 31 open on top, in which the scented cartridge 11 can be housed.

Furthermore, in the shown embodiment, the receptacle 31 is attached to the inside of the cover 29, whereby the tilting opening of the cover 29 hinged at the base makes it possible to access the open upper base 31a of the receptacle for taking out the cartridge 11 once exhausted and replace it with a fresh cartridge.

Advantageously, the apparatus 21 is capable of diffusing, into the environment, the essence contained in the cartridge 11 by means of a forced air movement caused by the tangential fan with which the apparatus is provided. According to the invention, the fan rotates at low speed so as to create a forced air flow sufficient to promote diffusion of the scent substance, without, however, excessively affecting the duration of the diffusion effect. In particular, the speed of the fan is maintained such as to make the air flow exiting the port 27 perceptible to the sense of smell, but substantially not to the sense of touch.

The invention as described and illustrated is susceptible to several variations and modifications falling within the same inventive principle.

1. A process for preparing a scent diffuser cartridge, comprising the steps of:
providing a quantity of granules of a material of natural mineral rock origin, capable of retaining a scent substance, wherein said material of natural mineral rock origin is vermiculite, perlite, hydroponic perlite, or a mixture thereof;
subjecting the material in the form of granules to sieving so as to obtain an over-sieve of granules having a size larger than that of the material usually available naturally and commercially, which is substantially free from dusts with a size such that they can penetrate deeply into the human respiratory system, and particularly to the level of the alveoli;
placing a predetermined quantity of said over-sieve granules into a container;
diffusing, into the container, a liquid scent substance in a quantity proportional to the quantity of granules present in the container;
closing the container;
letting the granules in the container stand for a predetermined time in order to cause the granules to absorb the scent substance diffused in the container;
checking whether any residues of liquid are present inside the container;
if, at the previous step, the presence of residues of liquid has been detected, letting again the granules in the container stand for a predetermined time and possibly repeating the previous step;
if, at the previous step, the absence of residues of liquid has been detected, providing, in a vessel, a mixture containing 60% to 90% granules having absorbed the scent substance and obtained with the previous steps, and 10% to 40% over-sieve granules;
letting the thus-obtained mixture stand;
providing a casing provided with an air-permeable porous wall;
placing a quantity of said mixture into the casing;
closing the casing;
packaging the cartridge in order to put it on the market.

2. (canceled)

3. The process according to claim 1, wherein the over-sieve material comprises granules having a maximum diameter size between 4 and mm.

4. The process according to claim 1, wherein the scent substance is diffused into the container by gravity in the form of rain or by nebulization.

5. The process according to claim 4, wherein the container is a vertically elongated cylindrical container, and wherein the step of diffusing the scent substance by gravity in the form of rain takes place from above by means of a strainer with holes having a diameter of about 1.0 mm, or through a nebulizing nozzle.

6. The process according to claim 1, wherein the granules of material in the container are let stand, at the end of the step of diffusing the scent substance, for a time proportional to the quantity of diffused substance, said time being one hour per each mL of liquid scent substance diffused in the container, at a temperature comprised in a predetermined range between 15 and 20 °C and in the dark.

7. The process according to claim 1, wherein the container containing the granules of material in which the scent substance has been diffused is turned upside down at the end of the standing step.

8. The process according to claim 1, wherein the step in which the granules of material in the container are let stand after the presence of liquid in the container has been detected for the first time after the standing step, is carried out for a time of 30 minutes per each mL of scent substance, for example essential oil/fragrance, diffused inside the vessel.

9. The process according to claim 1, wherein the casing provided with an air-permeable porous wall comprises a porous wall having a filtering capacity between 40 and 70 microns, such that the wall retains, inside the casing, particles with a diameter smaller than the selected range.

10. The process according to claim 1, wherein the mixture inside the vessel comprises 90% granules in which the scent substance has been diffused in the preceding steps of the process and 10% granules coming from the over-sieve and not subjected to the steps of diffusion of the scent substance.

11. The process according to claim 10, wherein the vessel comprises per each 10 g of mixture: 8 g of granules in which the scent substance has been diffused in the preceding steps of the process and 2 g of granules coming from the over-sieve and not subjected to the step of diffusing the scent substance.

12. The process according to claim 10, wherein the step of letting the mixture in the vessel stand is carried out for a time of four hours per each ten grams of compound obtained.

13.-14. (canceled)

15. A scent diffuser cartridge, produced by the process according to claim 1.

16. A diffuser apparatus (21) comprising a casing (23) and means for generating an air flow sucked from the environment outside the casing through a first port (25), which air flow passes through the casing and exits again to the outside through a second port (27) and which, while travelling inside the casing, hits a porous receptacle (31) in which a scented cartridge (11) made according to claim 1 is housed.

17. The apparatus according to claim 16, wherein the casing (23) of the apparatus (21) has a parallelepiped shape, and wherein the port (25) for the entrance of air and the port (27) for the exit of air are provided on two perpendicular, adjacent sidewalls (23a,23b) of the casing (23).

18. The apparatus according to claim 16 or 17, wherein a front wall (23c) of the casing (23) of the apparatus (21) comprises an opening that can be closed by means of a corresponding cover (29), said cover being hinged at its base to the casing (23) and having, on its inner surface, the cylindrical porous receptacle (31) open on top, in which the scented cartridge (11) can be housed.

19. The apparatus according to claim 17, wherein a front wall (23c) of the casing (23) of the apparatus (21) comprises an opening that can be closed by means of a corresponding cover (29), said cover being hinged at its base to the casing (23) and having, on its inner surface, the cylindrical porous receptacle (31) open on top, in which the scented cartridge (11) can be housed.

20. The process according to claim 3, wherein the over-sieve material comprises granules having a maximum diameter size between about 4 and 5 mm.

21. The process according to claim 3, wherein the scent substance is diffused into the container by gravity in the form of rain or by nebulization.

22. The process according to claim 21, wherein the container is a vertically elongated cylindrical container, and wherein the step of diffusing the scent substance by gravity in the form of rain takes place from above by means of a strainer with holes having a diameter of about 1.0 mm, or through a nebulizing nozzle.
